(19) United States
(12) Patent Application Publication
(10) Pub. No.: US 2026/0108646 A1
(43) Pub. Date: Apr. 23, 2026

(54) METHOD AND DEVICE FOR DISINFECTING AND/OR STERILISING OBJECTS

(71) Applicant: SCHWING TECHNOLOGIES GMBH, Neukirchen-Vluyn (DE)

(72) Inventor: Alfred SCHILLERT, Dorsten (DE)

(21) Appl. No.: 19/117,013

(22) PCT Filed: Sep. 29, 2023
(86) PCT No.: PCT/EP2023/077148

§ 371 Date: Mar. 28, 2025

(30) Foreign Application Priority Data
Sep. 30, 2022 (DE) 10 2022 125 301.5

(51) Int. Cl. A61L 2/20; A61L 2/24; A61L 2/26; A61L 101/46; A61L 103/50; A61L 103/65

(52) U.S. Cl. CPC A61L 2/20; A61L 2/24; A61L 2/26; A61L 2101/46; A61L 2103/50; A61L 2103/65; A61L 2202/121; A61L 2202/122; A61L 2202/14; A61L 2202/15

(57) ABSTRACT

The invention relates to a method for disinfecting and/or sterilizing objects, said method comprising the method steps of: placing an object in a process chamber that can be evacuated; evacuating the process chamber; introducing a treatment medium; carrying out the disinfection and/or sterilization process; and increasing the pressure in the process chamber. The invention also relates to: a software program for carrying out the method for disinfecting and/or sterilizing objects; and a device for disinfecting and/or sterilizing objects. The invention also relates to: a disinfected object comprising residues of the treatment medium and/or reaction products of the treatment medium and fewer than 2000 germs per m² on the surface of the disinfected object; and a sterilized object comprising residues of the treatment medium and/or reaction products of the treatment medium and a germ-free surface.

### METHOD AND DEVICE FOR DISINFECTING AND/OR STERILISING OBJECTS

The invention relates to a method for disinfecting and/or sterilizing objects with the method steps of placing an object in a process chamber that can be evacuated, evacuating the process chamber, introducing a treatment medium, carrying out the disinfection and/or sterilization process and increasing the pressure in the process chamber. The invention further relates to a software program for carrying out the method for disinfecting and/or sterilizing objects and to a device for disinfecting and/or sterilizing objects. The invention also relates to a disinfected object comprising residues of the treatment medium and/or reaction products of the treatment medium and fewer than 2000 germs per m² on the surface of the disinfected object, as well as a sterilized object with residues of the treatment medium and/or reaction products of the treatment medium and a germ-free surface.

### PRIOR ART

The peracetic acid-ethanol vacuum method is an established and cost-effective sterilization method for allogeneic bone transplants and also offers adequate antibacterial, antifungal and antiviral protection for allogeneic soft tissue transplants.

During disinfection and/or sterilization using PES under vacuum, the PES passes into the gas phase already at a low temperature in vacuo. This process is used to achieve the best possible disinfection and/or sterilization of the products at a harmless temperature.

Introduced in the eighties [Starke R, Hackensellner HA, von Versen R. Experimentelle Untersuchungen zur Entkeimung von Transplantationsmaterial mit Peressigsaure. Z Exp Chir Transplant Artificial Organs. 1984; 17(5):254-8.], to date more than 60,000 allogeneic bone transplants have been sterilized with peracetic acid (PES, CH3CO3H) and ethanol [131]. Several studies demonstrate the sufficient efficacy and safety of the procedure and could not demonstrate any adverse effect on the structural and biomechanical properties of the transplants [132, 133]. Viral protection was also confirmed during sterilization of soft tissue transplants such as Achilles tendon, cartilage and skin [152]. Furthermore, in an in vitro study, Scheffler et al. were unable to demonstrate any impairment of the structural and material properties of human BPTB allografts after sterilization using the peracetic acid-ethanol vacuum method [153]. Furthermore, in another in vitro study, PES showed neither cytotoxic nor pro-inflammatory effects on human patellar tendon allografts [98]. In this paper, the advantages of the peracetic acid-ethanol negative pressure method are applied for the disinfection and/or sterilization of objects, especially with internal surfaces, e.g. trousers or gloves.

### Object

It is therefore an object of the present invention to provide a method for disinfecting and/or sterilizing objects with which objects can be disinfected and/or sterilized reliably and cost-effectively.

It is also an object of the present invention to provide a device for disinfecting and/or sterilizing objects with which objects can be disinfected and/or sterilized reliably and cost-effectively. It is also an object of the present invention to provide a disinfected object. It is also an object of the present invention to provide a sterilized object.

The object is achieved with the method for disinfecting and/or sterilizing objects according to claim 1. Advantageous embodiments of the invention are provided in the dependent claims.

The method according to the invention for disinfecting and/or sterilizing objects has five method steps: In the first step, an object is placed on a holder in an evacuable process chamber of a device for disinfection and/or sterilization. For the purposes of this document, an object is a component that optionally has internal surfaces. An object is, for example, a piece of clothing particularly for use in laboratories and/or medical facilities. An object is therefore, for example, trousers, a jacket, a hood or a glove. The process chamber can be evacuated and opened or closed in a gas-tight manner. The volume of the process chamber can be variably selected depending on the dimensions of the object(s) to be disinfected and/or sterilized. The placement of an object can be done automatically, remotely and/or manually by a user. The process chamber may have suitable holders for this purpose.

The difference between disinfection and sterilization is that when an object is sterilized, all microorganisms, including their persistent forms (spores), are killed or irreversibly inactivated. The object is then generally free of microorganisms capable of reproduction; a maximum of one in 10⁶ germs may survive, i.e. only one in 1,000,000 treated objects would not be sterile. When an object is disinfected, pathogenic germs are also killed or irreversibly inactivated, but the number of germs to be eliminated is a factor of 10 smaller than with sterilization; the aim of disinfection is to reduce the germs by at least a factor of 10. Thanks to the disinfecting treatment, the object no longer poses a risk of infection.

In the second step, the process chamber is evacuated. Preferably, a vacuum pressure of less than 300 mbar, preferably less than 150 mbar, particularly preferably less than 50 mbar is generated in the process chamber.

In the third step, a treatment medium is introduced. For the purposes of this document, a treatment medium is peracetic acid or a peracetic acid-containing mixture with a peracetic acid content of at least 2 vol. %, preferably at least 3 vol. % and particularly preferably at least 4 vol. % peracetic acid. Alternatively, the starting substances of the peracetic acid can also be introduced into the process chamber to produce the peracetic acid in situ.

In the fourth step, the disinfection and/or sterilization process is carried out. For this purpose, the process parameters (pressure and temperature in the process chamber) are kept constant during an exposure time. This serves to eliminate germs, bacteria and viruses on the object. Only the pressure increases due to the evaporation of the PES. Optionally, the pressure change can be avoided by appropriate pumping out.

In the fifth step, the pressure in the process chamber is increased. When the pressure is increased, the process chamber contains a concentration of the treatment medium that is below the concentration of the treatment medium in the process chamber during the holding time of the process. Preferably, the concentration of the treatment medium during or after the increase in pressure in the process chamber is more than a factor of 10, particularly preferably more than a factor of 30, below the concentration of the treatment medium during the holding time.

In a further development of the invention, the object has an inner surface. The object has an outer side and an inner side opposite the outer side, which have corresponding outer and inner surfaces. The object is, for example, a piece of clothing, e.g. a pair of trousers, where the trouser legs each have an outer side with an outer surface and an inner side with an inner surface. The internal surfaces of an object can only be disinfected and/or sterilized using currently known methods with great expenditure of time.

In a further embodiment of the invention, when placing the object on the holder, an inner surface of the object is aligned with the holder. In a further embodiment of the invention, the object has an opening which leads to the inner surface of the object, wherein the holder is positioned such that parts of the holder are positioned in the opening. The parts of the holder that are positioned in the opening of the object allow access of the treatment medium to the opening of the internal surface and enable rapid and thorough disinfection and/or sterilization of the object.

In a further embodiment of the invention, the holder is positioned in the process chamber such that the opening is aligned with an inlet of the process chamber of the device for disinfection and/or sterilization. In particular, the holder ensures that the inner surface is accessible to the treatment medium and can be quickly and thoroughly disinfected and/or sterilized. This allows access of the treatment medium to the opening of the internal surface and enables rapid and thorough disinfection and/or sterilization of the object.

In a further development of the invention, the object has a flexible wall, wherein the flexible wall represents an inner surface of the object on one side, wherein the holder is positioned such that parts of the holder separate the flexible wall from other parts of the object. In particular, this ensures that the inner surface is accessible to the treatment medium and can be quickly and thoroughly disinfected and/or sterilized.

In an advantageous embodiment of the invention, the device for disinfection and/or sterilization has a control which is suitable and/or intended to execute a control program which controls the process parameters of the method for disinfection and/or sterilization. The control system is designed as a microcontroller with memory that contains a software program for controlling a process for disinfection and/or sterilization of objects.

In a further embodiment of the invention, the control is coupled to a sensor unit, a pump, the inlet and/or an outlet, wherein in order to carry out the method, the control of the pump, the inlet and/or the outlet is carried out as a function of the measured variables detected by the sensors of the sensor unit. To carry out the process, the pump, the inlet and/or the outlet are controlled depending on the measured variables recorded by the sensors of a sensor unit.

In a further embodiment of the invention, the introduction of the treatment medium takes place depending on the determined concentration of the treatment medium in the gas phase. The process chamber has a suitable sensor to determine the concentration. By introducing the treatment medium depending on its concentration in the process chamber, it is ensured that the concentration of the treatment medium is always correct in order to achieve rapid and thorough disinfection and/or sterilization of the object.

In a further development of the invention, the process chamber is preheated. In a further embodiment of the invention, the process chamber is preheated to the target temperature 35° C.-85° C., preferably 40° C.-75° C. and particularly preferably 50° C.-65° C. This speeds up the process of disinfecting and/or sterilizing objects.

In a further embodiment of the invention, the objects are packed in sterilization bags before being placed in the process chamber. In a further aspect of the invention, the sterilization bags are permeable to vapors. Sterilization bags, also known as autoclave bags or laminated bags, are used to protect items from contamination. They are made of a material that can withstand high temperatures and steam sterilization. This makes them ideal for use in hospitals, dental practices and other medical facilities. Sterilization bags provide a safe and efficient way to protect the object from contamination. With their puncture-proof construction and moisture-resistant material, they are ideal for use in any environment. Sterilization bags are puncture resistant so that they will not be damaged during the sterilization process. The material of the sterilization bag is moisture and bacteria resistant, so that the items are protected from contamination. The sterilization bags are lightweight and easy to store, making them convenient to use in any environment.

In a further embodiment of the invention, the process chamber is closed after the object has been placed in the process chamber. After closing, the process chamber is sealed gas-tight.

In a further embodiment of the invention, the process chamber is heated to process temperature after the process chamber is closed. Due to the placement of the object with the chamber open, the temperature in the process chamber drops, so it is heated again to the process temperature of 40° C.-65° C., preferably 50° C.-55° C.

In a further embodiment of the invention, the pressure in the process chamber is monitored. The process chamber is connected to a pressure sensor.

In an advantageous embodiment of the invention, the treatment medium is a PES solution. The PES disinfects chemically, it has an oxidizing effect on the microorganisms. It has a broad spectrum of activity, short reaction time and an irreversible effect. In addition, compared to other sterilization media, it is largely material-compatible, can be added in precise quantities and, due to the low concentration that can be used, is little to not damaging to the skin. Optionally, a mixture of peracetic acid, acetic acid, a strong oxidizing agent such as hydrogen peroxide and water is used. The acids and hydrogen peroxide are strong oxidizing agents that decompose microorganisms.

In a further embodiment of the invention, the process chamber is evacuated to a pressure of 1-50 mbar, preferably 10 mbar. In a further embodiment of the invention, the pressure in the process chamber after the introduction of the treatment medium is kept below the boiling curve of the treatment medium. In a further development of the invention, the pressure is kept below the boiling curve of the treatment medium during the entire duration of the disinfection and/or sterilization process. After the treatment medium is introduced, the treatment medium immediately changes into the gas phase. This ensures that even hard-to-reach regions of the object, especially internal surfaces of the object, are disinfected and/or sterilized.

In a further embodiment of the invention, after completion of the disinfection and/or sterilization process, the process chamber is flushed with ambient air, wherein the flushing preferably takes place at intervals. This ensures that the treatment medium has been completely removed from the process chamber and/or the object and cannot escape uncontrollably when the process chamber is opened, so that the operator or a bystander does not inhale the solution when opening the door or access. For the purposes of this document, a ventilation medium is air and technical gases. In a further aspect of the invention, ambient air is introduced during flushing up to a chamber pressure of 200-500 mbar.

In a further embodiment of the invention, after introducing ambient air up to a pressure of 200-500 mbar, the process chamber is evacuated again to 1-100 mbar, preferably 10-40 mbar, particularly preferably 20 mbar. This also ensures that the treatment medium has been completely removed from the process chamber and cannot escape uncontrollably when the process chamber is opened.

In a further embodiment of the invention, the method steps according to claims 23 and 24 are repeated once, preferably three times and particularly preferably five times. This also ensures that the treatment medium has been completely removed from the process chamber and cannot escape uncontrollably when the process chamber is opened.

In a further embodiment of the invention, after completion of the flushing process, the pressure in the process chamber is increased to ambient pressure. The disinfected products can then be removed from the process chamber.

In a further development of the invention, the treatment medium is discharged. The treatment medium is thus removed from the process chamber.

In a further embodiment of the invention, a neutralization medium is introduced to neutralize the treatment medium. PES is an acid, so a base is optionally used as a neutralization medium. This also ensures that the treatment medium has been completely removed from the process chamber and cannot escape uncontrollably when the process chamber is opened.

The object is also achieved by means of the software program for carrying out the method according to one or more of claims 1 to 28.

The software program according to the invention is suitable for carrying out the method for controlling processes by means of defined values and/or curves for pressure, injection quantities (with defined concentrations), temperature and PES concentration in the gas phase in the chamber as well as by means of material and/or product information, their quantity, size, shape, etc., their degree of contamination and pollution and/or type of contamination (spores, viruses, etc.). The software program uses the device's hardware to disinfect and/or sterilize objects.

The object is further achieved by means of the device for disinfection and/or sterilization of objects. Advantageous embodiments of the invention are provided in the dependent claims.

The device according to the invention for disinfecting and/or sterilizing objects has an evacuable process chamber. The process chamber can be evacuated and opened or closed in a gas-tight manner. The volume of the process chamber can be variably selected depending on the dimensions of the object(s) to be disinfected and/or sterilized.

The device further comprises a first connection and/or first reservoir for a treatment medium and a first inlet in the process chamber, wherein the first inlet is connected to a first connection and/or the first reservoir for the treatment medium. A treatment medium is preferably peracetic acid or a peracetic acid-containing solution with a peracetic acid content of at least 2 vol. %, preferably at least 3 vol. % and particularly preferably at least 4 vol. % peracetic acid. Alternatively, the starting substances of the peracetic acid can also be introduced into the process chamber to produce the peracetic acid in situ.

The device also has a second connection and/or second reservoir for a ventilation medium and a second inlet in the process chamber, wherein the second inlet is connected to the second connection and/or the second reservoir for the ventilation medium and/or the first connection and/or the first reservoir. The ventilation medium is preferably air, but technical gases, e.g. noble gases, are also possible.

Furthermore, the device comprises a pump connected to one or more outlets, and a controller for controlling the first inlet, the second inlet, the first connection, the second connection and/or the outlet. Optionally, the controller controls the temperature and/or the pressure and/or the injection quantity and/or the concentration of a treatment medium in the chamber. Furthermore, the duration and number of process phases and cycles can be controlled by the controller.

The device further comprises a holder which is suitable for receiving an object. By means of the holder, the object is positioned and fixed in the process chamber in such a way that an inner surface of the object is optionally aligned with the holder, whereby the holder aligns the inner surface with the process chamber. The holder can be designed differently depending on the type and dimensions of the object.

In a further embodiment of the invention, the device has an outlet for discharging the treatment medium from the process chamber. The outlet is optionally connected to a filter and a pump through which the treatment medium can be discharged from the process chamber.

In a further embodiment of the invention, the controller is coupled to the sensor unit, the pump, the first inlet, the second inlet and/or the outlet. The controller is designed as a microcontroller with memory that contains a software program for controlling a process for disinfection and/or sterilization of objects. To carry out the process, the pump, the inlet and/or the outlet are controlled depending on the measured variables detected by the sensors of a sensor unit.

In a further embodiment of the invention, the holder is suitable for holding objects with an inner surface such that the inner surface is accessible to the process chamber. In particular, the holder ensures that the inner surface is accessible to the treatment medium and can be quickly and thoroughly disinfected and/or sterilized.

In a further embodiment of the invention, a part of the holder can be positioned in an opening of the object, wherein the opening of the object leads to the inner surface of the object. The part of the holder that is positioned in the opening of the object thus allows access of the treatment medium to the opening of the internal surface and enables rapid and thorough disinfection and/or sterilization of the object.

In a further embodiment of the invention, the holder is positioned in the process chamber such that the opening and/or the inner surface of the object can be aligned with the inlet of the device for disinfection and/or sterilization. This allows access of the treatment medium to the opening of the internal surface and enables rapid and thorough disinfection and/or sterilization of the object.

The object is also achieved by means of the disinfected object with an internal surface. An advantageous embodiment of the invention is provided in the dependent claim.

The disinfected object with an internal surface has residues of the treatment medium and/or reaction products of the treatment medium of less than 2000 germs per m² on the surface of the disinfected object. The disinfected object according to the invention has a contamination with germs on its surface of less than 2000 germs per m², preferably less than 1000 germs per m² and particularly preferably less than 500 germs per m², which corresponds to a germ reduction of 84% to 99.9%. In a further development of the invention, the disinfected object contains residues of peracetic acid or residues of reaction products of peracetic acid.

The disinfected object is preferably an object from the following groups: masks, safety goggles/visors, surgical textiles, disposable protective gowns, lightweight hoods (blower filter system), blower filter devices, protective overalls (emergency services/fire brigade), surgical shoes, surgical magnifying glasses, ventilation tubes, masks, bags, Pari-Boy nebulizers for inhalation therapy (with electronics), clinical thermometers, full face masks, lung regulators, compressed air breathing apparatus, but also electronic devices such as radios and a smartphone. The material of the disinfected object is: PP, PET/PETG, nylon, PU/PU foams, PVC foam, ABS, elastane, EPDM, silicone, rubber or metal, and/or a composite of the previously mentioned materials.

The object is also achieved by the sterilized object with an internal surface. Advantageous embodiments of the invention are provided in the dependent claims.

The sterilized object with an internal surface has residues of the treatment medium and/or reaction products of the treatment medium and a germ-free surface. The sterilized object according to the invention has a contamination with bacteria on its surface of 0 if the probability of a surviving germ is less than 1:1,000,000 (<=10⁻⁶ per unit of the object).

In a further aspect of the invention, the inner surface has residues of the medium and/or reaction products of the treatment medium and is germ-free. In a further development of the invention, the sterilized object and the inner surface have residues of peracetic acid or residues of reaction products of peracetic acid. In an optional further development, the concentration of the treatment medium and/or reaction products of the treatment medium is below 0.1 ml/m² or below 0.32 ml/m² and in a particularly preferred embodiment below 0.05 ml/m² or below 0.16 ml/m².

The sterilized object is preferably an object from the following groups: masks, safety goggles/visors, surgical textiles, disposable protective gowns, lightweight hoods (blower filter system), blower filter devices, protective overalls (emergency services/fire brigade), surgical shoes, surgical magnifying glasses, ventilation tubes, masks, bags, Pari-Boy nebulizers for inhalation therapy (with electronics), clinical thermometers, full face masks, lung regulators, compressed air breathing apparatus, but also electronic devices such as radios and a smartphone. The material of the disinfected object is: PP, PET/PETG, nylon, PU/PU foams, PVC foam, ABS, elastane, EPDM, silicone, rubber or metal, or a composite of the previously mentioned materials.

Exemplary embodiments of the device and method according to the invention for disinfecting and/or sterilizing objects are shown schematically in the drawings in a simplified manner and are explained in more detail in the following description.

In the drawings:

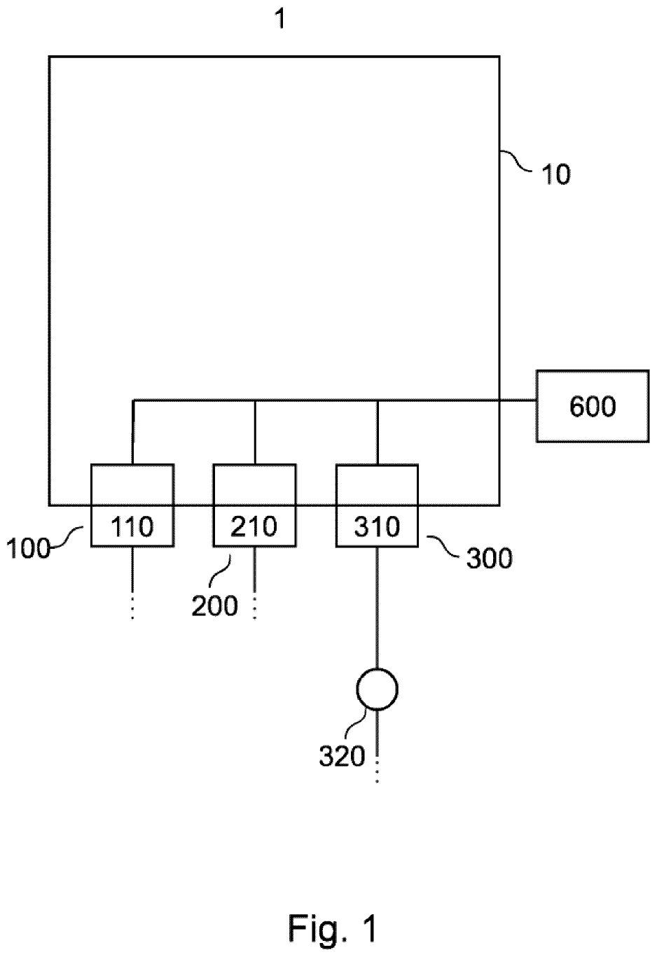

FIG. 1 shows a device for disinfecting and/or sterilizing objects

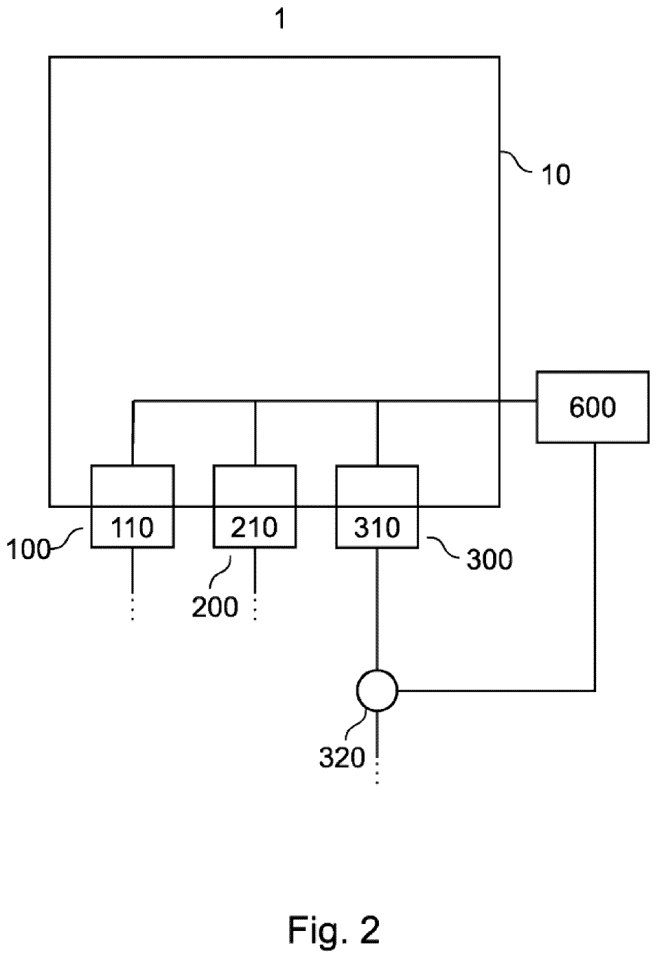

FIG. 2 shows a device for disinfecting and/or sterilizing objects, control connected to vacuum pump

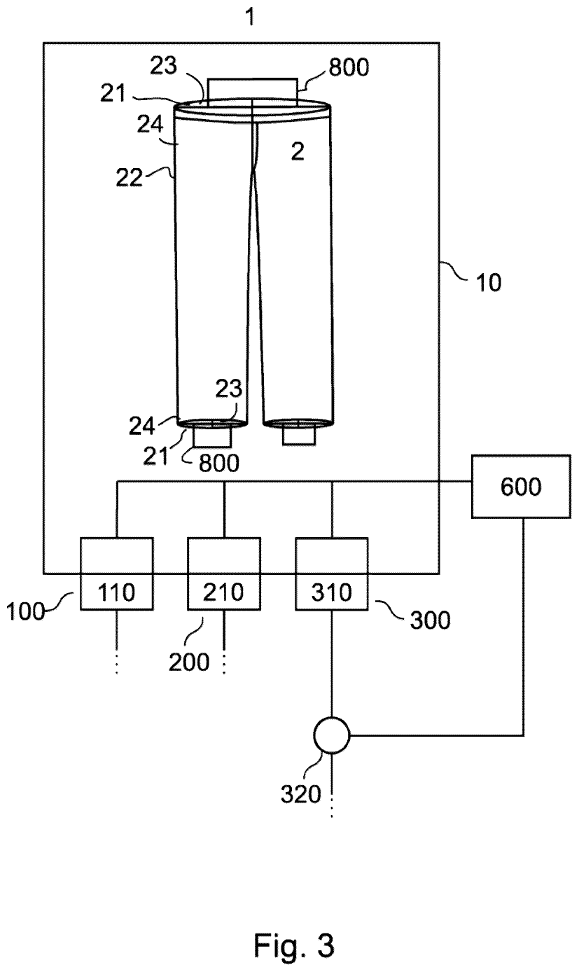

FIG. 3 shows an exemplary embodiment of an object arranged in the device

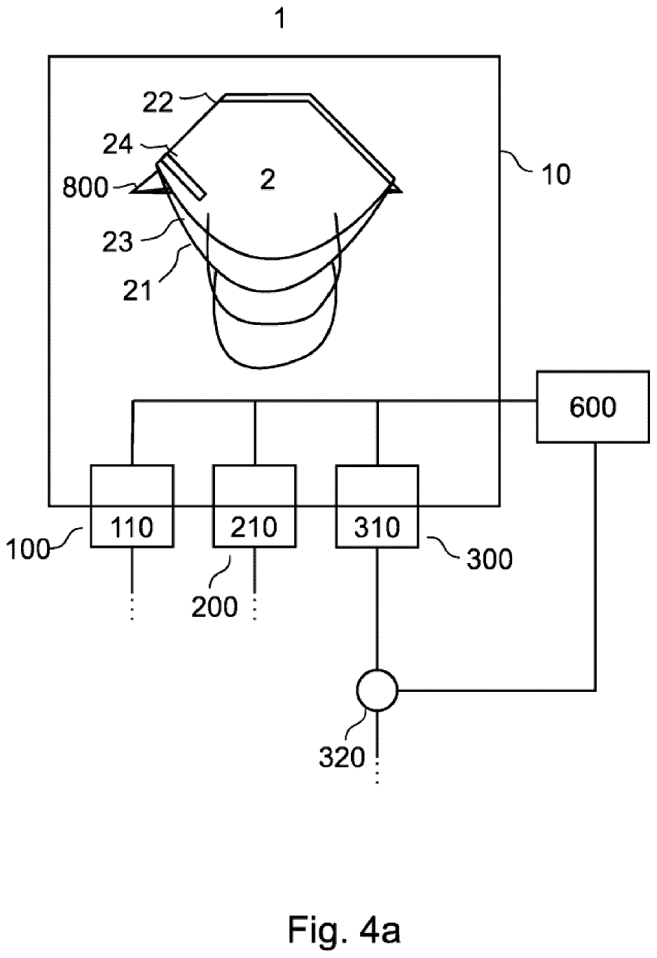

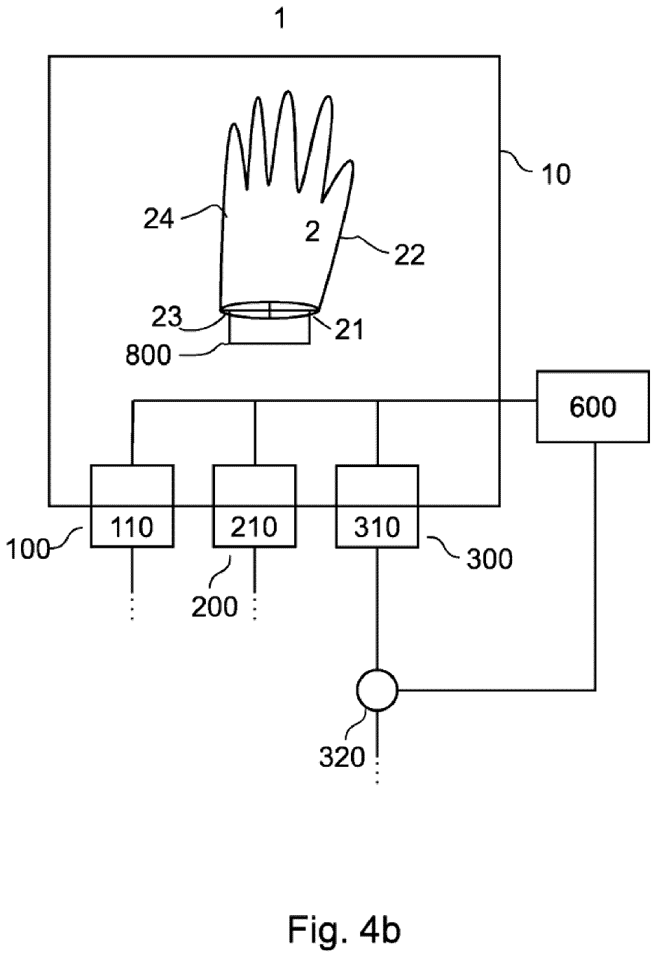

FIG. 4a shows an exemplary embodiment of an object arranged in the device

FIG. 4b shows an exemplary embodiment of an object arranged in the device

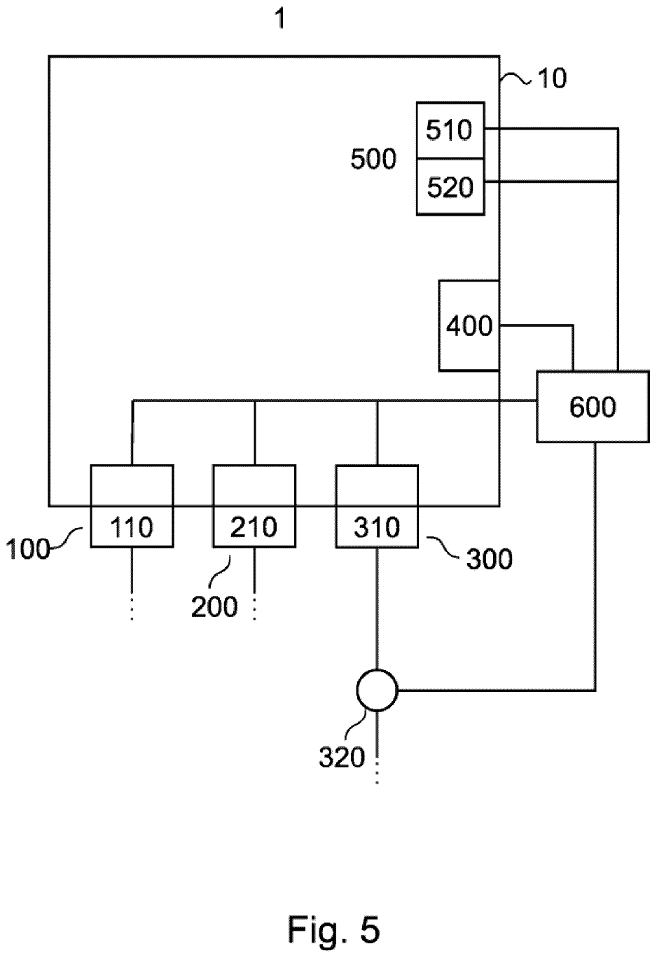

FIG. 5 shows a device for disinfecting and/or sterilizing objects, pressure and temperature sensors

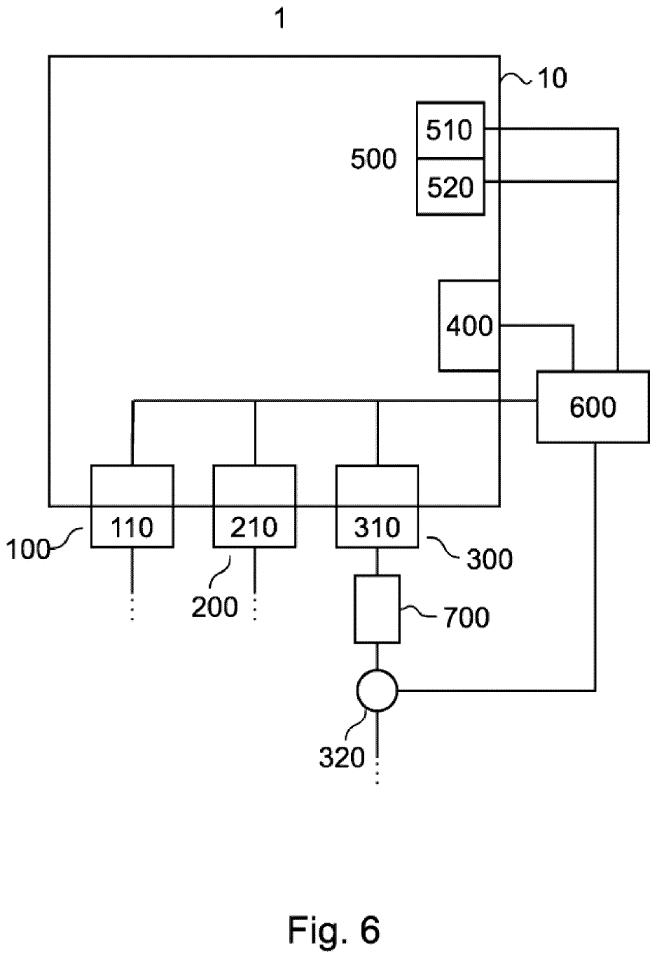

FIG. 6 shows a device for disinfecting and/or sterilizing objects, pressure and temperature sensors, HEPA filter in medium discharge

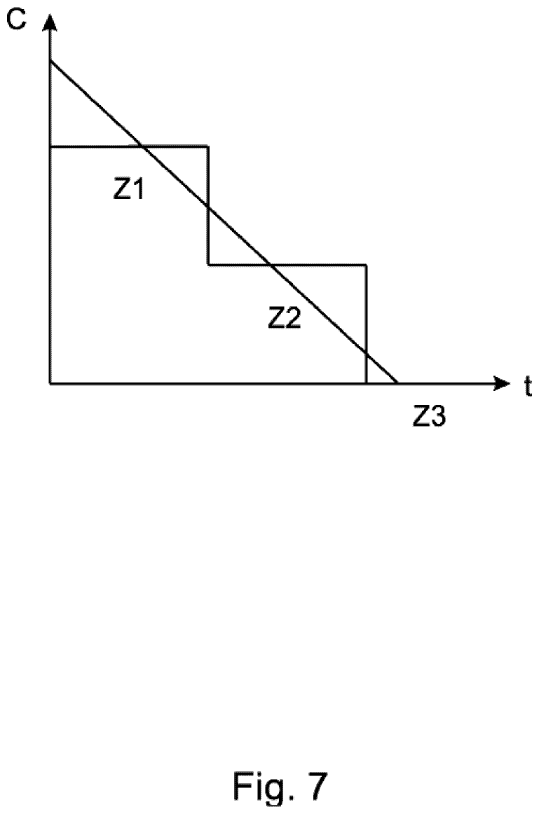

FIG. 7 shows an exemplary embodiment of the method for disinfection and/or sterilization of objects

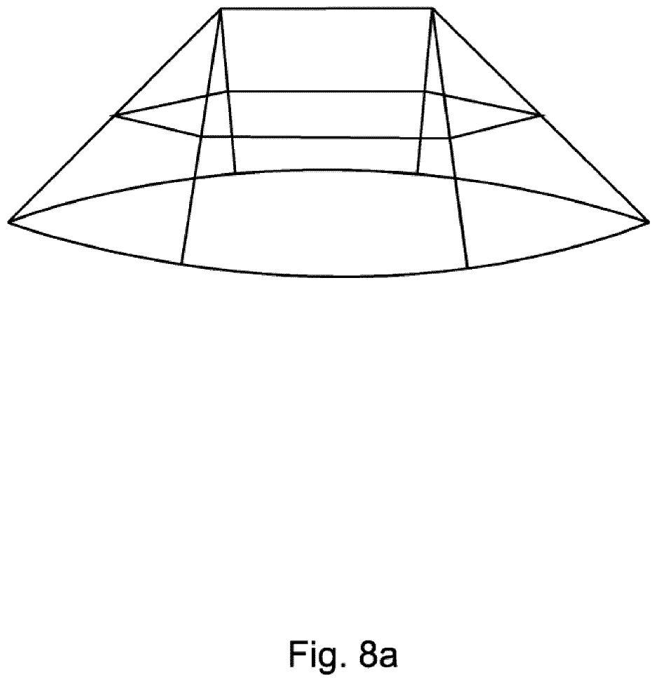

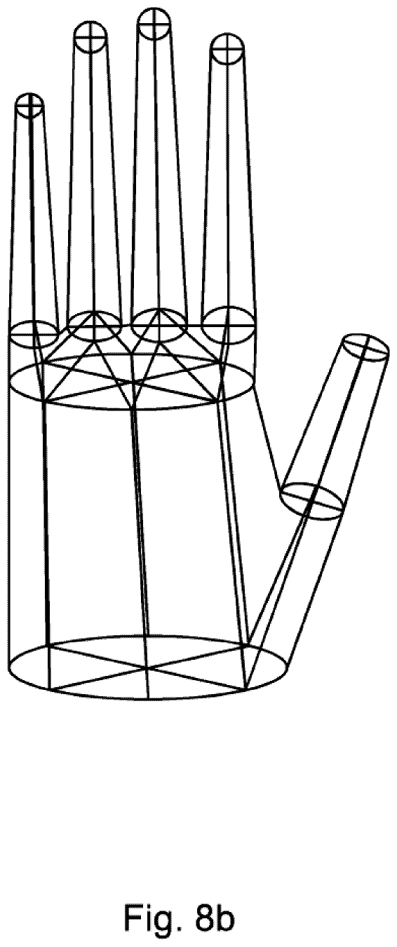

FIG. 8a shows an exemplary embodiment of a holder

FIG. 8b shows an exemplary embodiment of a holder

FIG. 1 and FIG. 2 show exemplary embodiments of the device 1 according to the invention for disinfecting and/or sterilizing objects 2. The device 1 has the process chamber 10, which is designed to be evacuated and has a volume of 50 liters, wherein 100 liters is also possible. For evacuation, the vacuum pump 320, designed as a rotary pump, is connected via the outlet connection 310 to the outlet 300 of the process chamber 10 (FIG. 1). The vacuum pump 320 is connected to the controller 600 (FIG. 2). The outlet 300 can be opened or closed and is connected to the control 600. The device 1 has the medium discharge for discharging the treatment medium from the process chamber 10.

The process chamber 10 has a first inlet 100 which is connected to a reservoir for the treatment medium via a first connection 110. Also connected to the controller 600 is the second inlet 200, which is connected to the reservoir for the ventilation medium via the second connection 210. The inlets 100, 200, like the outlet 300, can be opened or closed via valves, controlled by the controller 600.

For disinfection and/or sterilization of objects 2, the object 2 is placed on a holder 800 in the process chamber 10. Optionally, the object 2 is packed in a vapor-permeable sterilization bag before it is placed in the process chamber 10 in order not to contaminate the object 2 during and after disinfection and/or sterilization and to place a contaminated object 2 easily and safely in the process chamber 10.

The process chamber 10 is then sealed gas-tight and evacuated to 20 mbar using the vacuum pump 320. The treatment medium is then introduced by the controller 600 sending a signal to the first inlet 100 to open the first inlet 100. The first inlet 200 is connected to the reservoir for the treatment medium. In all exemplary embodiments shown here, the treatment medium is a mixture of peracetic acid and water. A treatment medium is preferably peracetic acid or a peracetic acid-containing mixture with a peracetic acid content of at least 2 vol. %, preferably at least 3 vol. % and particularly preferably at least 4 vol. % peracetic acid. Alternatively, the starting substances of the peracetic acid can also be introduced into the chamber to produce the peracetic acid in situ. Due to the low pressure in the process chamber 10, the solution is brought into the gaseous state in order to fill the process chamber 10 and carry out the oxidation process on the objects to be disinfected. Different concentrations of PES are provided for the process in order to be able to design the disinfection and/or sterilization process flexibly. The first inlet 100 is closed again after the treatment medium has been introduced.

For disinfection and/or sterilization of the objects 2, the object 2 in the process chamber 10 is left in the process chamber 10 for between 5 minutes and 120 minutes at constant pressure and temperature and exposed to the PES atmosphere. Thereafter, the pressure in the process chamber 10 is increased by the controller 600 sending a signal to the second inlet 200 to open the second inlet 200. The second inlet 200 is connected to the reservoir for the ventilation medium. The ventilation medium in all embodiments is air, but technical gases, e.g. noble gases, are also possible. When the pressure is increased, the process chamber 10 contains a concentration of the treatment medium that is below the concentration of the treatment medium in the process chamber 10 during the holding time of the process. Preferably, the concentration of the treatment medium during or after the increase in pressure in the process chamber 10 is more than a factor of 10, particularly preferably more than a factor of 30, below the concentration of the treatment medium during the holding time. The process chamber 10 can be opened and the object 2 removed.

A further exemplary embodiment of the device 1 according to the invention for disinfecting and/or sterilizing objects 2 is shown in FIG. 3. The device 1 shown here corresponds to the device shown in the previous embodiment (see FIG. 2), in this embodiment, an object 2 to be disinfected and/or sterilized is arranged on a suitable holder 800 in the process chamber 10. In this embodiment, the object 2 is a pair of trousers as part of a surgical textile, the material of the trousers 2 is a composite of rubber and silicone. The material of the object 2 forms a flexible wall 22 which is gas-tight. On the outside, the object 2 has the outer surface 24, and on the inside it has the inner surface 23. At a first end, the object 2 has an opening 21 (waistband), on the side opposite the first end, the object 2 has two openings 21 which open into the trouser legs. The openings 21 lead to the inner surface 23 of the object 2.

The holder 800 itself is designed such that the object 2 arranged on the holder 800 is aligned with the inner surface 23 relative to the holder 800. Parts of the holder 800 are positioned in the respective openings 21. The object 2 is arranged on the holder 800 such that the flexible wall 22 is arranged such that the openings 21 and thus the inner surface 23 are accessible for a gas flow. When arranging the holder 800 in the process chamber 10, the holder 800 is positioned such that at least one, in this exemplary embodiment two openings 21—the openings 21 which open into the trouser legs—are aligned with the inlet 100. In this way, the inner surface 23 is easily accessible for the treatment medium. The object 2 is disinfected and/or sterilized by means of the method described in FIG. 1 and FIG. 2.

FIG. 4 shows further exemplary embodiments of the design and arrangement of holders 800 in the process chamber 10. The device 1 shown here corresponds to the device 1 shown in the previous embodiment (see FIG. 2, FIG. 3). The object 2 to be disinfected and/or sterilized is a surgical mask (FIG. 4a) or a surgical glove (FIG. 4b). These differently formed objects 2 are arranged on holders 800 which are also formed differently from each other (see FIG. 8).

The arrangement of the object 2 on the holder 800 is such that the respective openings 21 of the object 2 allow access to the inner surface 23 of the flexible wall 22. The inner surface 23 faces the holder 800, the outer surface 24 faces away from the holder 800. In addition, the holder 800 is designed and arranged in the process chamber 10 in such a way that the individual parts and/or regions of the object 2 arranged on the holder 800 do not touch each other, i.e. the flexible wall 22 is at a distance from all parts of the object 2. In the embodiment shown in FIG. 4b, for example, the individual fingers of the glove 2 are arranged at a distance from one another on the holder 800. As a result, both the outer surface 22 and the inner surface 23 are easily accessible to the treatment medium. When arranging the holder 800 in the process chamber 10, the holder 800 is also positioned such that the opening 21 is aligned with the inlet 100. The object 2 is also disinfected and/or sterilized by means of the method described in FIG. 1 and FIG. 2.

FIG. 5 and FIG. 6 show exemplary embodiments of the device 1 according to the invention for disinfecting and/or sterilizing objects 2. The devices 1 shown here correspond to the device shown in the previous embodiment (see FIG. 2 to FIG. 6), only the process chamber 10 has a heating device 400, which is designed as a resistance heater and is connected to the controller 600, and a sensor unit 500 with two sensors 510, 520, which are also connected to the controller 600 (FIG. 5). In a further embodiment, the device 1 additionally has a HEPA filter 700, which is arranged in the line between pump 320 and outlet 300 (FIG. 6).

The sensors 510, 520 are arranged in the process chamber 10 and record physical measured values in the process chamber 10. The sensor 510 is a temperature sensor for recording and monitoring the temperature within the process chamber 10. The sensor 520 is a pressure sensor for recording and monitoring the pressure within the process chamber 10.

For disinfection and/or sterilization of objects 2, after placing the object 2 in the process chamber 10 and evacuating it, the temperature in the process chamber 10 is increased by means of the heating device 400 after closing the process chamber 10, to a temperature of 50° C. to 55° C., alternatively to a temperature of 40° C. to 65° C. To shorten the heating process, the process chamber 10 can be preheated before placing the object 2 in the process chamber 10.

In the next step, the treatment medium (PES solution) is introduced into the process chamber 10 via the first inlet 100. In an advantageous development of the invention, the injection quantity and/or the concentration of a treatment medium in the process chamber 10 is controlled by means of the controller 600. Furthermore, the duration and number of process phases and cycles are controlled. The PES solution immediately begins to boil and the pressure in the process chamber 10 increases. By means of the controller 600, the pressure and temperature in the process chamber 10 are controlled during a part of the process in such a way that the boiling curve of the PES solution is at least partially not exceeded in order to prevent condensation of the PES solution. This state is kept constant for part of the process duration. This serves to eliminate germs, bacteria and viruses on the object 2. Only the pressure in the process chamber 10 increases due to the evaporation of the PES. Optionally, this pressure change can be avoided by pumping out the pressure using pump 320.

By means of the temperature sensor 510, the controller 600 continuously detects the temperature in the process chamber 10 during the process and regulates the temperature in the process chamber 10 via the heating device 400. The pressure in the process chamber 10 is detected with the pressure sensor 520 and also regulated by the controller 600 by either opening the inlet 100 or reducing the pressure in the chamber via the outlet 300 and the pump 320.

In a further embodiment, the sensor device 500 may comprise a chemical sensor. By means of the chemical sensor, the concentration of the treatment medium (PES solution) in the process chamber 10 is constantly detected and monitored by the controller 600. This makes it possible to record the consumption of the treatment medium during the procedure and also to determine whether the disinfection and/or sterilization process is complete. If the concentration of the treatment medium is low, the disinfection and/or sterilization process is not yet complete; the concentration of the treatment medium in the sample chamber 10 may need to be increased. For this purpose, the first inlet 100 is opened by means of the inlet connection 110 under the control of the controller 600 and further treatment medium is fed into the sample chamber 10. If a high concentration of the treatment medium remains, the disinfection and/or sterilization process is complete.

After the end of the process, the process chamber 10 is flushed at intervals with the ventilation medium (air). The ventilation medium is preferably germ-free, i.e. no microorganisms and/or viruses are contained in the ventilation medium that are larger than 0.45 micrometers, preferably 0.22 micrometers. For this purpose, ambient air is let into the process chamber 10 via the second inlet 200 controlled by the controller 600 up to a pressure in the process chamber 10 of 200 mbar to 500 mbar, preferably 300 mbar. The pressure is then pumped down to 1 mbar to 100 mbar, preferably approx. 40 mbar, particularly preferably approx. 20 mbar. This flushing process is carried out one to five times, preferably three times, to ensure that the treatment medium has been completely removed from the process chamber 10, cannot escape uncontrollably when the process chamber 10 is opened, and the operator or a bystander does not inhale the solution when opening the door or access. In a further development, the ventilation medium is introduced until the concentration of the treatment medium in the chamber is between 0.5 g/m and 4 g/m³; preferably between 1 g/m³ and 3 g/m³; particularly preferably between 1.5 g/m³ and 2.5 g/m³. Optionally, a neutralization medium, e.g. a gaseous base, can be introduced before the flushing process with the ventilation medium in order to neutralize the treatment medium. The air inlet or air discharge via an outlet 300 is preferably carried out via HEPA filters. The HEPA filter 700 serves to protect the environment and people from germs when a vacuum is generated at the start of the process and also to protect object 2 from contamination.

FIG. 7 shows an embodiment of the method according to the invention for disinfection and/or sterilization of objects 2, which is carried out in three cycles. The graph shows the spore/virus/bacteria concentration C on the inner and outer surfaces of object 2 (ordinate) over time t (abscissa).

After carrying out the method with the device 1 according to the invention, the object 2 has traces of the treatment medium (PES) and/or reaction products of the treatment medium with other substances on its inner surface 23 and outer surface 24. In an optional further development, the concentration of the treatment medium and/or reaction products of the treatment medium is below 0.1 ml/m² or below 0.32 ml/m² and in a particularly preferred embodiment below 0.05 mUm² or below 0.16 ml/m².

After carrying out the method with the device 1 according to the invention, the disinfected object 2 according to the invention has a contamination with germs on its inner surface 23 and outer surface 24 of less than 2000 germs per m², preferably less than 1000 germs per m² and particularly preferably less than 500 germs per m² of surface, which corresponds to a germ reduction of 84% to 99.9%. The sterilized object 2 according to the invention has a contamination with germs on its surface of 0 if the probability of a surviving germ is less than 1:1,000,000 (<=10⁻⁶ per unit of the object 2).

The disinfection and sterilization process is usually carried out depending on the object 2 to be sterilized/disinfected, the boundary conditions and predeterminable requirements according to processes specifically optimized in the control 600 and the sequence. For this purpose, different cycles, intervals and process parameters can be set via a software program stored in the controller 600 or, if required, individually via the UI of the controller 600. For example, if the germ load of object 2 is high, different cycles of disinfection and/or sterilization may have to be run, since the PES solution is used up at a high germ load, so that the subsequent addition of PES may be necessary. This step will be carried out in cycles, among other things. During the process, the treatment medium (PES) will be pumped out and added, or ventilation will take place. In addition, the cycle progression is also suitable for small volumes of both the process chamber 10 and the object 2 as well as difficult-to-reach and complex geometries of the object 2.

FIG. 8 shows exemplary embodiments of different holders 800 for holding different objects 2. A holder 800 is optionally manufactured individually for each object type (e.g. trousers, mask, glove). In these exemplary embodiments, a holder 800 for a surgical mask (see FIG. 4a) and a holder 800 for a surgical glove (see FIG. 4b) are shown. In these embodiments, the holders 800 are made of a wire mesh, wherein the wire is a stainless steel that is acid and temperature resistant. The wire mesh allows the treatment medium to access the inner surface 23 of the object 2 arranged on the holder. Other possible materials for the holder 800 are structurally stable plastics, e.g. ABS, silicone, nylon.

TABLE 1
Overview of validation tests for disinfection
of spore strips SAL 10⁵

| | PES in | Concentration of | | |
|---|---|---|---|---|
| | solution | PES in process | Process | Biological Result |
| | [%] | chamber [g/m³] | duration[min] | #not sterile/#tested |
| 1 | 6 | 3 | 240 | 0/2 |
| 2 | | | 120 | 0/1 |
| 3 | | 1.7 | 150 | 0/2 |
| 4 | 4 | 2 | 120 | 0/1 |
| 5 | | 1.9 | 120 | 0/2 |
| 6 | | | 90 | 0/2 |
| 7 | | | 60 | 0/4 |
| 8 | | | 45 | 0/1 |
| 9 | | | 40 | 0/2 |
| 10 | | | 30 | 0/2 |
| 11 | | | 20 | 0/2 |
| 12 | | | 15 | 0/1 |
| 13 | | | 10 | 0/1 |
| 14 | | | 5 | 0/1 |
| 15 | | 1.7 | 20 | 0/1 |
| 16 | | | 15 | 0/1 |
| 17 | | 1.5 | 240 | 0/2 |
| 18 | | | 150 | 0/2 |
| 19 | | 1.3 | 20 | 0/1 |
| 20 | | | 15 | 0/1 |
| 21 | | 0.94 | 60 | 0/5 |
| 22 | | | 40 | 0/1 |
| 23 | | | 30 | 0/2 |
| 24 | | | 20 | 0/2 |
| 25 | | | 15 | 0/1 |
| 26 | | | 10 | 0/1 |
| 27 | | | 5 | 0/1 |
| 28 | 4 | 0.47 | 60 | 0/1 |
| 29 | | | 30 | 0/1 |
| 30 | | | 15 | 0/1 |
| 31 | | | 10 | 0/1 |
| 32 | | | 5 | 0/1 |

TABLE 2
Overview of validation tests for disinfection of laboratory
utensils sprinkled with spore suspension SAL 10⁶

| | PES in | Concentration of | | |
|---|---|---|---|---|
| | solution | PES in process | Process | Biological Result |
| | [%] | chamber [g/m³] | duration[min] | #not sterile/#tested |
| 5 | 4 | 1.9 | 120 | 0/4 |
| 7 | | | 60 | 0/4 |
| 8 | | | 45 | 0/4 |
| 12 | | | 15 | 0/4 |
| 13 | | | 10 | 0/4 |
| 14 | | | 5 | 0/4 |
| 15 | | 1.7 | 20 | 0/4 |
| 16 | | | 15 | 0/4 |
| 19 | | 1.3 | 20 | 0/4 |
| 20 | | | 15 | 0/4 |
| 21 | | 0.94 | 60 | 0/4 |
| 22 | | | 40 | 0/4 |
| 23 | | | 30 | (pipette) 1/8 |
| 24 | | | 20 | 0/8 |
| 25 | | | 15 | (pipette) 1/4 |
| 26 | | | 10 | 0/4 |
| 27 | | | 5 | (pipette) 1/4 |
| 28 | 4 | 0.47 | 60 | 0/4 |
| 29 | | | 30 | 0/4 |
| 30 | | | 15 | (Eppi) 1/4 |
| 31 | | | 10 | (pipette) 1/4 |
| 32 | | | 5 | (mask, pipette) 2/4 |

When an object is subject to disinfecting treatment, pathogenic germs are also killed or irreversibly inactivated, but the number of germs to be eliminated is a factor of 10 smaller than with sterilization; the aim of disinfection is to reduce the germs by at least a factor of 10⁵. Thanks to the disinfecting treatment, the object 2 no longer poses a risk of infection.

The effectiveness of the disinfecting treatment is defined by the probability of the presence of life-threatening microorganisms. This probability is expressed by the sterility assurance level (SAL); a SAL of at least 10⁻⁵ defines disinfection, a SAL of at least 10⁻⁶ defines sterilization, i.e. the lower the SAL value, the higher the safety.

The disinfectant and sterilizing agent used in device 1 presented here is peracetic acid (PES) in a solution diluted with water. The PES disinfects chemically, it has an oxidizing effect on the microorganisms. It has a broad spectrum of activity, short reaction time and an irreversible effect. In addition, compared to other sterilization media, it is largely material-compatible, can be added in precise quantities and, due to the low concentration that can be used, is little to not damaging to the skin.

The validation tests of the “disinfection and sterilization device” were carried out using spores of the bacterium Geobacillus stearothermophilis. Bacterial spores exhibit a very high resistance (C) to chemical disinfection and/or sterilization. If bacterial spores can be successfully disinfected in the processes, it can be assumed that the process will also successfully disinfect microorganisms with moderate resistance (A) such as lipophilic viruses, vegetative bacteria, fungi (including spores), organisms such as E. faecium, S. aureus, P. aeruginosa, A. niger as well as microorganisms with high resistance (B) such as mycobacteria, hepatitis B virus and hydrophilic viruses such as the polio virus.

The validation tests were initially carried out with bio-indicator spore strips 6×36 mm, onto each of which a colony forming unit (CFU) of 10⁵ of Geobacillus stearothermophilus was inoculated. The spore strips were each packed in a sterilization bag. During the tests, different process times and different concentrations of PES were tested. All tests were carried out at a chamber temperature of 50-55° C.

In all spore strips tested at different PES concentrations and different processing times, no growth or proliferation of the spores could be detected afterwards. In addition, untreated test samples were included in each experiment. This ensured that the samples were inoculated with spores

In addition to the validation tests with the spore strips SAL 10⁵, various disposable products were also sprinkled with a spore suspension. These included a vaccination loop, a disposable 3 ml pipette, a micro reaction vessel with lid (Eppi) and a piece of nose and mouth mask. The spore suspension was an alcoholic solution and was permeated with Geobacillus stearothermophilus in a CFU/ml of at least 10⁶. Due to the small amount, the samples were each dripped with 2 drops of the suspension (in the case of the pipette and the Eppi, the suspension was dripped into them) and then placed in a sterilization bag. One drop corresponds to 0.05 ml, so two drops correspond to a unit of measurement of 0.1 ml. Therefore, the CFU per object is approximately 10′.

The samples were tested with some of the spore strip tests listed above. After treatment, the swabs of the samples, analogous to the spore strips, were placed in a nutrient solution in the external laboratory for seven days and then examined for possible growth of spores.

In various tests, spore growth was observed after seven days. This is due to the relatively low PES concentration in the process chamber as well as the short process duration.

The disinfection or sterilization system according to the invention is designed in such a way that different concentrations of PES can be specifically set in the vacuum of the process chamber. The combination of special injection technology, adapted vacuum process and process cycles ensures that the disinfecting and/or sterilizing medium mixture is safely distributed even in inaccessible places and that the PES has a very high effectiveness against biogenic contamination even at low temperatures of 40° C. to 65° C., preferably 50° C. to 55° C.

The disinfection or sterilization system according to the invention, including the disinfection and/or sterilization processes and associated device technology, thus has the following decisive advantages: high effectiveness, low-temperature process (also suitable for thermolabile polymers), low operating costs, short process times, individual adaptation of the disinfection and/or sterilization programs to different requirements, very good scalability (from mobile table-top devices to mobile room-sized systems) and general use of a disturbance-free, safe process.

Due to the combination of the above advantages this innovative disinfection and sterilization system—in contrast to the methods already used—is therefore ideally suited for numerous known and new areas of application. For example, in infection control in small to large healthcare and nursing facilities, where the entire spectrum from compact tabletop devices to large systems is required. The same applies to the areas of application of fire services and disaster control, where the systems and equipment must be particularly robust and reliable and, in some cases, mobile.

In the future, the PES processing system will include in particular the following process-specific and application-related innovations:
a. Novel, very broadly applicable chemical low-temperature disinfection and/or sterilizationsystem
   The PES as a reactive component can either be produced in low concentrations during in-situ mixing of the different starting materials or adjusted to higher concentrations by targeted mixing from a storage container in the system. PES reacts with the bacteria, viruses, spores, etc., which are killed quickly and efficiently. The individual components (hydrogen peroxide and acetic acid) for the in-situ production of PES are globally available, inexpensive chemicals that can be handled safely while taking the necessary occupational safety measures into account.
   Gaseous PES is characterized by a high sterilizing effect. The use of gaseous PES in an automated vacuum process of a closed system ensures both hazard-minimized handling and safe action.
   Standardized, validatable method (no manual disinfection, e.g. wipe disinfection)
   The starting materials (hydrogen peroxide and acetic acid) can be easily removed quantitatively from the treatment system and treated products, so that the treated protective clothing can be reused without endangering personnel.
b. This mobile, customized disinfection and/or sterilizationdevice technology can be used for almost all application and operating conditions for rapid PES disinfection and/or sterilization, even on site in the event of major disasters.
c. Integrated monitoring and documentation system for the disinfection and/or sterilizationprocess, i.e. regarding the protective effect after the preparation of the PPE
d. The special design and process control of this chemical-physical treatment process (PES, vacuum, temperature) in combination with the specially adapted treatment system provides short treatment times which are sometimes 8-25 minutes. These treatment times depend on the processing objective (disinfection or sterilization, if necessary), the PPE materials or material combinations, and the surfaces and geometries).
e. The disinfection and/or sterilization of PPE is possible in the new processing system within the sterilization bag, thus ensuring safe and contamination-free handling.

### LIST OF REFERENCE NUMERALS

1 device for disinfecting and/or sterilizing objects
10 process chamber
100 first inlet
110 first connection
200 second inlet
210 second connection
300 outlet
310 outlet connection
320 pump
400 heating device
500 sensor unit
510 temperature sensor
520 pressure sensor
600 controller
700 HEPA filter
800 holder
2 object
21 opening
22 flexible wall
23 inner surface
24 outer surface

1. A method for disinfecting and/or sterilizing objects (2) with the following steps:
placing an object (2) in an evacuable process chamber (10) of a device (1) for disinfection and/or sterilization,
evacuating the process chamber (10),
introducing a treatment medium,
carrying out the disinfection and/or sterilization process,
increasing the pressure in the process chamber (10).

2. The method for disinfecting and/or sterilizing objects (2) according to claim 1,
characterized in that
the object (2) has an inner surface (23).

3. The method for disinfecting and/or sterilizing objects (2) according to claim 1,
characterized in that
when placing the object (2) on a holder (800), an inner surface (23) of the object (2) is aligned with the holder (800).

4. The method for disinfecting and/or sterilizing objects (2) according to claim 1,
characterized in that
the holder (800) aligns the inner surface (23) with the process chamber (10).

5. The method for disinfecting and/or sterilizing objects (2) according to claim 1,
characterized in that
the object (2) has an opening (21) which leads to the inner surface (23) of the object (2),
wherein the holder (800) is positioned such that parts of the holder (800) are positioned in the opening (21).

6. The method for disinfecting and/or sterilizing objects (2) according to claim 1,
characterized in that
the holder (800) is positioned in the process chamber (10) such that the opening (21) is aligned with an inlet (100, 200) of the process chamber (10) of the device (1) for disinfection and/or sterilization.

7. The method for disinfecting and/or sterilizing objects (2) according to claim 1,
characterized in that
the object (2) has a flexible wall (22),
wherein the flexible wall (22) on one side represents an inner surface (23) of the object (2),
wherein the holder (800) is positioned such that parts of the holder (800) space the flexible wall (22) apart from other parts of the object (2).

8. The method for disinfecting and/or sterilizing objects (2) according to claim 1,
characterized in that
the device (1) for disinfection and/or sterilization has a controller (600) which is suitable and/or provided to execute a control program which controls the process parameters of the method for disinfection and/or sterilization.

9. The method for disinfecting and/or sterilizing objects (2) according to claim 1,
characterized in that
the controller (600) is coupled to a sensor unit (500), a pump (320), the inlet (100) and/or an outlet (300),
wherein, to carry out the method, the control of the pump (320), the inlet (100) and/or the outlet (300) is carried out as a function of the measured variables detected by the sensors of the sensor unit (500).

10. The method for disinfecting and/or sterilizing objects (2) according to claim 1,
characterized in that
the introduction of the treatment medium is dependent on the determined concentration of the treatment medium.

11. The method for disinfecting and/or sterilizing objects (2) according to claim 1,
characterized in that
the process chamber (10) is preheated.

12. The method for disinfecting and/or sterilizing objects (2) according to claim 1,
characterized in that
the process chamber (10) is preheated to the target temperature 35° C.-85° C., preferably 40° C.-75° C. and particularly preferably 50° C.-65° C.

13. The method for disinfecting and/or sterilizing objects (2) according to claim 1,
characterized in that
the objects (2) are packed in sterilization bags before being placed in the process chamber (10).

14. The method for disinfecting and/or sterilizing objects (2) according to claim 1,
characterized in that
the sterilization bags are permeable to vapors.

15. The method for disinfecting and/or sterilizing objects (2) according to claim 1,
characterized in that
the process chamber (10) is closed after the object (2) has been placed in the process chamber (10).

16. The method for disinfecting and/or sterilizing objects (2) according to claim 1,
characterized in that
the process chamber (10) is heated to process temperature after the process chamber (10) has been closed.

17. The method for disinfecting and/or sterilizing objects (2) according to claim 1,
characterized in that
the pressure in the process chamber (10) is monitored.

18. The method for disinfecting and/or sterilizing objects (2) according to claim 1,
characterized in that
the treatment medium is a PES solution or a PES-containing solution.

19. The method for disinfecting and/or sterilizing objects (2) according to claim 1,
characterized in that
the process chamber (10) is evacuated to a pressure of 1-80 mbar, preferably 10 mbar.

20. The method for disinfecting and/or sterilizing objects (2) according to claim 1,
characterized in that
the pressure after the introduction of the treatment medium is kept partially below the boiling curve of the treatment medium.

21. The method for disinfecting and/or sterilizing objects (2) according to claim 20,
characterized in that
the pressure is kept below the boiling curve of the treatment medium during the entire duration of the disinfection and/or sterilization process.

22. The method for disinfecting and/or sterilizing objects (2) according to claim 1,
characterized in that
after completion of the disinfection and/or sterilization process, the process chamber (10) is flushed with ambient air,
wherein flushing is preferably carried out at intervals.

23. The method for disinfecting and/or sterilizing objects (2) according to claim 1,
characterized in that
during flushing, ambient air is introduced up to a chamber pressure of 200-500 mbar.

24. The method for disinfecting and/or sterilizing objects (2) according to claim 1,
characterized in that
after introducing ambient air up to a pressure of 200-500 mbar, the process chamber (10) is evacuated again to 1-100 mbar, preferably 10-40 mbar, particularly preferably 20 mbar.

25. The method for disinfecting and/or sterilizing objects (2) according to claim 22,
characterized in that
during flushing, ambient air is introduced up to a chamber pressure of 200-500 mbar and after introducing ambient air up to a pressure of 200-500 mbar, the process chamber (10) is evacuated again to 1-100 mbar, preferably 10-40 mbar, particularly preferably 20 mbar are repeated once, preferably three times and particularly preferably five times.

26. The method for disinfecting and/or sterilizing objects (2) according to claim 1,
characterized in that
after completion of the flushing process, the pressure in the process chamber (10) is increased to ambient pressure.

27. The method for disinfecting and/or sterilizing objects (2) according to claim 1,
characterized in that
the treatment medium is discharged.

28. The method for disinfecting and/or sterilizing objects (2) according to claim 1,
characterized in that
a neutralization medium is introduced to neutralize the treatment medium.

29. A software program for carrying out the method according to claim 1.

30. A device (1) for disinfecting and/or sterilizing objects (2), comprising
an evacuable process chamber (10),
a first connection (110) and/or first reservoir for a treatment medium
a first inlet (100) in the process chamber (10); wherein the first inlet (100) is connected to a first connection (110) and/or the first reservoir for the treatment medium
a second connection (210) and/or second reservoir for a ventilation medium
a second inlet (200) in the process chamber (10), wherein the second inlet (200) is connected to the second connection (210) and/or the second reservoir for the ventilation medium and/or the first connection (110) and/or the first reservoir.
a pump (320) connected to one or more outlets (300)
a controller (600) for controlling the first inlet (100), the second inlet (200), the first connection (110), the second connection (210) and/or the outlet (300).
a holder (800).

31. The device (1) for disinfecting and/or sterilizing objects (2) according to claim 30,
characterized in that
the device (1) has an outlet (300) and a media discharge for discharging the treatment medium from the process chamber (10).

32. The device (1) for disinfecting and/or sterilizing objects (2) according to claim 31,
characterized in that
the controller (600) is coupled to the sensor unit (500), the pump (320), the first inlet (100), the second inlet (200) and/or the outlet (300).

33. The device (1) for disinfecting and/or sterilizing objects (2) according to claim 30,
characterized in that
the holder (800) is suitable for receiving objects (2) with an inner surface (23) such that the inner surface (23) is accessible to the process chamber (10).

34. The device (1) for disinfecting and/or sterilizing objects (2) according to claim 33,
characterized in that
a part of the holder (800) can be positioned in an opening (21) of the object (2), wherein the opening (21) of the object (2) leads to the inner surface (23) of the object (2).

35. The device (1) for disinfecting and/or sterilizing objects (2) according to claim 34,
characterized in that
the holder (800) is positioned in the process chamber (10) such that the opening (21) and/or the inner surface (23) of the object (2) can be aligned with the inlet of the device (1) for disinfection and/or sterilization.

36. A disinfected object (2) with an internal surface (23) with residues of the treatment medium and/or reaction products of the treatment medium of less than 2000 germs per m² on the surface (23, 24) of the disinfected object (2).

37. The disinfected object (2) with an inner surface (23) according to claim 36,
characterized in that
the disinfected object (2) has residues of peracetic acid or residues of reaction products of peracetic acid.

38. The disinfected object (2) with an inner surface (23) according to claim 36,
characterized in that
the inner surface (23) has residues of the medium and/or reaction products of the medium and less than 2000 germs per m² on the inner surface (23) of the object (2).

39. A sterilized object (2) with an inner surface (23) with residues of the treatment medium and/or reaction products of the treatment medium and a germ-free surface (23, 24).

40. The sterilized object (2) with an inner surface according to claim 39,
characterized in that
the inner surface (23) has residues of the treatment medium and/or reaction products of the treatment medium and is germ-free.

41. The sterilized object (2) with an inner surface (23) according to claim 39,
characterized in that
the sterilized object (2) has residues of peracetic acid or residues of reaction products of peracetic acid.
